(19) United States
(12) Patent Application Publication
(10) Pub. No.: US 2019/0120818 A1
(43) Pub. Date: Apr. 25, 2019

(54) WIRELESS RFID-BASED ANEMIA SENSING SYSTEM

(71) Applicant: Massachusetts Institute of Technology, Cambridge, MA (US)

(72) Inventors: Sanjay E. Sarma, Lexington, MA (US); Rahul Bhattacharyya, Portland, ME (US); Christina D. Swanson, Cambridge, MA (US)

(73) Assignees: Massachusetts Institute of Technology, Cambridge, MA (US); Diagnostics for All, Inc., Salem, MA (US)

(21) Appl. No.: 16/093,194

(22) PCT Filed: Apr. 14, 2017
(86) PCT No.: PCT/US2017/027689

§ 371 Date: Oct. 12, 2018

Related U.S. Application Data
(60) Provisional application No. 62/322,890, filed on Apr. 15, 2016.

(51) Int. Cl. G01N 33/49; G01N 27/02; H01Q 1/22; B01L 3/00

(52) U.S. Cl. CPC G01N 33/49; G01N 27/023; B01L 2300/021; B01L 3/502; B01L 2300/0663; H01Q 1/2283

(57) ABSTRACT

An RFID-based anemia detection sensor that integrates a paper-based diagnostic device with a passive Ultra High Frequency (UHF) RFID tag. Differences in red blood cell (RBC) count in a patient's blood manifests itself as a controlled time-dependent change in the tag's signal response. In one embodiment, the sensor is capable of reliably differentiating between blood having 20, 30, 40 and 50 percent RBC concentration by volume, which is indicative of anemic vs. healthy blood. In another embodiment, the sensor is read using standard RFID equipment allowing for large-scale automated screening of blood specimens.

### FIELD OF THE INVENTION

The invention relates generally to diagnostic medical devices and specifically to devices for detecting anemia.

### RELATED APPLICATIONS

This application claims priority to and the benefit of provisional application Ser. No. 62/322,890, filed on Apr. 15, 2016, which is herein incorporated in its entirety by reference.

### BACKGROUND OF THE INVENTION

Radio frequency identification (RFID) has recently seen use in applications that streamline logistics and reduce medical errors in the healthcare industry. RFID systems are capable of wirelessly interrogating and extracting ID from tags deployed on physical assets in the hospital setting. RFID systems afford automated, non line-of-sight object identification at large scale and are currently being used to track expensive equipment, monitor patient and staff motion and ensure correct drug distribution.

Recently there have been attempts to use RFID tags for applications beyond object identification, such as pervasive sensing. RFID tags have been used in several applications ranging from implants to DNA characterization and enzyme detection. Where cost is a driving factor, designs making use of passive RFID tags are particularly appealing. The advent of paper-based analytical devices presents several opportunities in passive RFID based-sensing.

Paper-based analytical devices (μ-PADs) are devices consisting of channels created by patterning paper with hydrophobic materials. Biological and chemical reagents are then added to the channels to create different tests. While many of these tests have a colorimetric readout and are read by eye, novel methods for data acquisition and management are needed to maintain assay objectivity, increase sensitivity and obtain quantitative measurements. There is also a need to automate the process of output readout and scale the functionality to multiple tests running in parallel.

### SUMMARY OF THE INVENTION

Hematocrit is defined as the percentage of red blood cells (RBCs) by volume in the blood and can range from 20% to 60%. Normal values of hematocrit vary according to a person's age, sex, pregnancy status and altitude. Healthy adult males typically have a hematocrit ranging from 42% to 54%. Healthy adult females have a hematocrit ranging from 38% to 46%, and normal babies and children may range from 29% to 68%. Hematocrit values below these normal values for the specified population may indicate that the individual is suffering from anemia. Hence, an assay to rapidly screen for anemia should differentiate hematocrit values between 20% and 40%, while producing a stable signal for hematocrits levels above 50%.

The assay for measurement of the hematocrit percentage in whole blood is based on the principal that, in a porous structure, plasma will separate more slowly from blood samples with higher hematocrits. This is thought to occur because red blood cells, when placed on a porous membrane, are pulled into pores by capillary force and become entrapped if the pore size is smaller than the diameter of the cell. As more blood cells become trapped, they block some of the pores, reducing the open area for plasma to filter through the membrane and thus, reducing the plasma flow speed in the membrane.

The present invention relates to an RFID-based anemia detection sensor designed by coupling a μ-PAD device, seeded with a fixed volume of a patient's blood, with an RFID tag. Differences in red blood corpuscle concentration in the blood manifest themselves as controlled changes in the RFID tag backscatter signal response. By observing these changes at the RFID reader, it is possible to discriminate between the blood of a healthy and anemic patient.

In one aspect, the invention relates to a method of measuring hematocrit. In one embodiment, the method includes the steps of providing an assay device including an RFID tag; a micro-PAD defining a sample port and a channel, the micro-PAD in electrical communication with the RFID tag; measuring, using an RFID reader the change of impedance of the RFID tag over time; and determining the hematocrit of the sample in response to the time change of impedance.

In another aspect the invention relates to a system for measuring the hematocrit of a sample. In one embodiment, the system includes an assay device including: an RFID tag;

a micro-PAD defining a sample port and a channel, the micro-PAD in electrical communication with the RFID tag; and an RFID reader, wherein the RFID reader measures the change of impedance of the RFID tag over time; and determines the hematocrit of the sample in response to the time change of impedance.

Yet another aspect the invention relates to a wireless blood anomaly detection system. In one embodiment, the system includes: an RFID tag including an RFID chip and an RFID antenna; and a blood probe including: a substrate defining a port in fluid communication with a channel, the channel including a metallic strip and a ground plane, wherein the metallic strip is in electrical communication with the RFID antenna, wherein the port is configured to accept a blood sample, and wherein the channel has an impedance which changes as the blood sample moves from the port and through the channel, and an RFID reader generating a carrier signal and configured to monitor the change in back scatter signal of the carrier signal, as reflected back by the RFID tag, as blood moves down the channel.

In another embodiment, the RFID reader measures the time it takes for blood to move down the channel in response to a change in back scatter frequency and generates a hematocrit value in response to the time of blood movement. In yet another embodiment, the RFID reader measures the time it takes for blood to move down the channel in response to a change in signal strength and generates an hematocrit value in response to the time of blood movement.

Still yet another aspect of the invention relates to a wireless blood anomaly detection sensor. In one embodiment, the sensor includes an RFID tag comprising an RFID chip and an RFID antenna; and a blood probe comprising: a substrate defining a port in fluid communication with a channel, the channel including a metallic strip and a ground plane, wherein the metallic strip is in electrical communication with the RFID antenna, wherein the port is configured to accept a blood sample, and wherein the channel has an impedance which changes as the blood moves from the port and through the channel

Another aspect of the invention relates to a method for blood anomaly detection. In one embodiment, the method includes the steps of providing an RFID tag comprising an RFID chip and an RFID antenna; and a blood probe. In another embodiment, the blood probe includes: a substrate defining a port in fluid communication with a channel, the channel including a metallic strip and a ground plane, wherein the metallic strip is in electrical communication with the RFID antenna, wherein the port is configured to accept a blood sample, and wherein the channel has an impedance which changes as the blood sample moves from the port and through the channel; generating a carrier signal with an RFID reader; and monitoring with the RFID reader, the change in back scatter of the carrier signal as blood moves down the channel.

In yet another embodiment, the backscatter measurement includes measuring a change in signal strength. In still yet another embodiment, the backscatter measurement includes measuring a change in the frequency response of the RFID tag received by the RFID reader.

Yet another aspect of the invention relates to a wireless fluid component detection system. In one embodiment, the system includes: an RFID tag comprising an RFID chip and an RFID antenna; and a fluid probe including: a substrate defining a port in fluid communication with a channel, the channel including a metallic strip and a ground plane, wherein the metallic strip is in electrical communication with the RFID antenna, wherein the port is configured to accept a fluid sample, and wherein the channel has an impedance which changes as the fluid sample moves from the port and through the channel, and an RFID reader generating a carrier signal and configured to monitor the change in back scatter signal of the carrier signal, as reflected back by the RFID tag, as fluid moves down the channel.

In another embodiment, the RFID reader measures the time it takes for a fluid sample to move down the channel in response to a change in RFID tag back scatter frequency and generates a component concentration value in response to the time of fluid movement. In yet another embodiment, the RFID reader measures the time it takes for a fluid sample to move down the channel in response to a change in signal strength and generates a component concentration value in response to the time of fluid movement.

Still yet another aspect of the invention relates to a method for fluid component detection. In one embodiment, the method includes the steps of providing an RFID tag comprising an RFID chip and an RFID antenna; and a fluid probe. In another embodiment, the fluid probe includes: a substrate defining a port in fluid communication with a channel, the channel including a metallic strip and a ground plane, wherein the metallic strip is in electrical communication with the RFID antenna, wherein the port is configured to accept a fluid sample, and wherein the channel has an impedance which changes as the fluid sample moves from the port and through the channel; generating a carrier signal with an RFID reader; and monitoring with the RFID reader, the change in back scatter of the carrier signal as fluid moves down the channel.

In another embodiment, the backscatter measurement comprises measuring a change in signal strength. In yet another embodiment, the backscatter measurement comprises measuring a shift or change in the frequency response of the RFID tag received by the RFID reader.

Another aspect of the invention relates to a wireless fluid component detection sensor. In one embodiment, the sensor includes an RFID tag comprising an RFID chip and an RFID antenna; and a fluid probe including: a substrate defining a port in fluid communication with a channel, the channel including a metallic strip and a ground plane, wherein the metallic strip is in electrical communication with the RFID antenna, wherein the port is configured to accept a fluid sample, and wherein the channel has an impedance which changes as the fluid moves from the port and through the channel.

### BRIEF DESCRIPTION OF THE DRAWINGS

The structure and function of the invention can be best understood from the description herein in conjunction with the accompanying figures. The figures are not necessarily to scale, emphasis instead generally being placed upon illustrative principles. The figures are to be considered illustrative in all aspects and are not intended to limit the invention, the scope of which is defined only by the claims.

FIG. 1 depicts an embodiment of a channel assay device used for characterization of RFID tag performance, in which: (W) is the hydrophobic wax that helps shape the flow channel (C); (S) is a 5 mm sample port where fluid is added and an arrow is provided showing the direction of flow of fluid along the channel.

FIG. 2 depicts an embodiment of a microstrip probe having a μ-PAD channel.

FIG. 3 depicts the dependence of probe impedance on fluid flow.

FIG. 4 is a photograph of an embodiment of a probe in straight channel connected to an RFID tag.

FIG. 5 is a photograph of an embodiment of a Stepped Impedance Resonator (SIR), with μ-PAD paper superstrate connected to a Vector Network Analyzer (VNA) for S21 power measurement.

FIG. 6 is a graph of the frequency variation of S21 of the SIR filter when no superstrate is added and a graph of the frequency variation when wet and dry μ-PAD paper is added as a superstrate.

FIG. 7 is a table of εr and tan(δ) values for the embodiment of the μ-pad channel that generated the graph of frequency variation shown in FIG. 6.

FIG. 8 is a graph of ΔP_{BS|reader} computed for varying probe lengths. This term is the difference in signal response of the sensor when the channel is completely dry vs. completely wet for varying probe lengths.

FIGS. 9A, 9B, 9C, 9D, and 9E are graphs of RFID tag backscatter signal drop with different fluids and paper materials. The individual graphs depict various RFID tags attached to paper-based assay devices constructed of nitrocellulose (FIGS. 9A-9C), Whatman 1 (FIG. 9D) and Whatman 4 (FIG. 9E) membranes. In FIG. 9A, 10 μL of DI H20; in FIG. 9B 10 μL of 1X PBS; and in FIG. 9C 10 μL of plasma were added to the nitrocellulose membranes. 20 μL of plasma was added to the Whatman membranes (FIGS. 9D and 9E).

FIG. 10 is a graph of RFID tag backscatter power change with differential channel wetting. RFID tags were attached to nitrocellulose assays and 10 μL of plasma was added to each device. The power change was determined as the plasma flowed in the channel underneath the probe. Averages and standard errors are computed by repeating each test eight times

FIG. 11 is an embodiment of a curved channel design that utilizes regions of sensitivity in the copper probe.

FIG. 12 depicts a channel with demarcators of stages S1-S5.

FIGS. 13A, 13B, and 13C are graphs of the measurement of hematocrit percentage in a blood sample. FIG. 13A is a representative graph depicting power measurements versus time for blood with 30% hematocrit. The graph may be divided into five stages labeled S1-S5 as shown in FIG. 12. FIG. 13B is a graph of the average flow times of blood with different hematocrit percentages as determined utilizing endpoints 1 and 2 with remote RFID sensing. In FIG. 13B, the tests were repeated four times for each hematocrit percentage level. FIG. 13C is a graph of the comparison of flow times as determined visually and utilizing remote RFID sensing with endpoint.

FIG. 14 is a photograph of an embodiment of a sensor with plasma flow at stage S3. The plasma flow and the zone where plasma separates from the RBCs are labeled.

### DESCRIPTION OF A PREFERRED EMBODIMENT

The last decade has seen several advances in novel low-cost, RFID-based sensing. Researchers have achieved this either by interfacing the RFID's tag IC with sensor electronics or by using the tag's antenna as a sensor. The latter approach often uses electromagnetically responsive smart materials or background changes to an RFID tag to cause a controlled change in tag signal parameters, such as backscatter signal strength or response frequency. There have been several case studies where researchers have developed RFID devices to sense parameters like temperature alarms, humidity, volatile organics, and pH.

Researchers have even used RFID tag-sensors in several bio-medical applications. For instance, one design uses implantable RFID tags in body sensors. Another design uses an RFID tag as a wireless front end in a DNA sequence characterization device. In this design, different DNA sequences cause different changes in light intensity that can be detected by a photo-sensor. The photo-sensor data is then conveyed wirelessly to an RFID reader by interfacing the sensor with an RFID tag. Fluorescence intensity in chemiluminescent immunoassays for detecting thyroid-stimulating hormones have also utilized RFID sensors.

Paper-based analytical devices (μ-PADs) are being developed for a wide range of functions, from rapid point-of-care measurement of liver enzyme levels to routine measurement of heavy metal contamination in reservoir water. One recently created μ-PAD measured femtomolar quantities of an enzyme based upon flow time. In this assay, an area of hydrophobic polymer was applied to both an experimental and control channel, initially retarding flow. If a blood sample contained a certain enzyme, hydrogen peroxide produced in the experimental channel switched the hydrophobic polymer to a hydrophilic polymer, allowing fluid to flow through the channel more rapidly. As the fluid reached the end of each channel, it picked up dried green dye in the channel and appeared as a green circle on top of the assay. The time difference for fluid to flow through the experimental channel versus the control channel was calculated using visual interpretation of the appearance of the green dye circles and a timer. This time difference directly correlated to the original enzyme concentration. While the concept of a timing assay demonstrates promise—having a high sensitivity and tolerance to volume, temperature and humidity changes—the assay requires the user to continually monitor the experimental and control areas for color change, recording the time taken for each event.

To overcome the issues of the monitoring of the timing assay and to scale the functionality to multiple tests running in parallel, the present invention relates to an RFID tag-sensor that can remotely monitor the flow of microliter volumes of fluid in paper membranes. As fluid flows down a channel it selectively detunes the RFID tag sensor. This manifests itself as a sharp, predictable drop in the strength of the backscatter signal from the RFID tag. By wirelessly monitoring the time at which the signal drops, remotely monitoring of paper-based timing assays is accomplished.

To demonstrate the efficacy of the flow sensing RFID tag for a paper-based timing assay the present invention uses a timing assay to screen for anemia based on hematocrit levels in whole blood. According to the World Health Organization, anemia afflicts 47% of preschool-age children and 41% of pregnant women worldwide, impairing cognitive and physical development. Anemia, which is characterized by a reduced ability of the circulatory system to transport oxygen through the body, is clinically defined by either a low hematocrit or a low hemoglobin level. As the hematocrit percentage directly affects the ability of paper membranes to separate plasma and the volume of plasma that may be separated, it is indirectly measured by timing the movement of plasma along the length of a channel. The screening test for anemia relies upon measuring the time for fluid to propagate to two different areas of a paper-based device. This assay is able to easily and automatically differentiate blood samples with hematocrit levels ranging from 20% to 50%, thus successfully being able to discriminate between blood samples of anemic and non-anemic patients.

A novel RFID tag-sensor was designed and characterized using a paper-based microfluidic channel Subsequently, an optimized paper-based microfluidic assay for hematocrit measurement based on this channel was developed and integrated with the RFID tag.

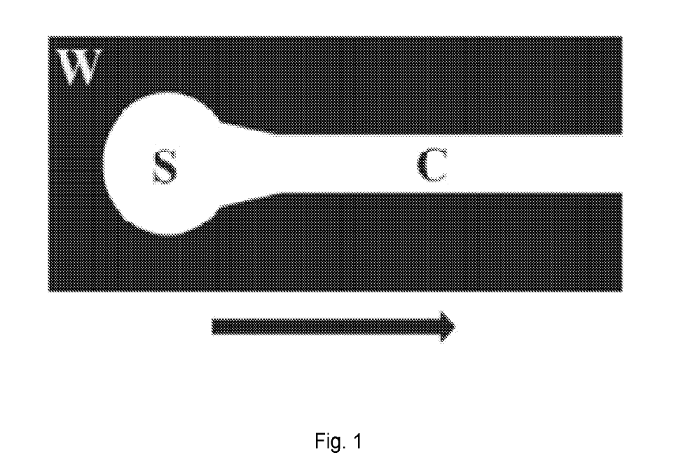

In one embodiment, a straight channel paper microfluidic assay device was developed for integration with the RFID tag. In one embodiment, the channel included a 5 mm diameter sample port (S) where a specified volume of fluid is added. Contiguous to the sample port is a 20×2 mm hydrophilic channel (C) (FIG. 1). To create the hydrophobic barriers surrounding the hydrophilic channels, a layer of wax (W) was printed on top of pre-shrunk paper. The wax was evenly coated using oven at 400° F. The straight channel design was manufactured from several papers commonly used to develop paper-based microfluidic assays, including nitrocellulose membrane (EMD Millipore, Billerica, Mass.), Whatman 1 and Whatman 4 paper. All assays were stored at room temperature until used. Other papers and membranes may be used.

The paper-based microfluidic assay device is then used as a microstrip probe. The probe's impedance characteristics are significantly altered as the fluid propagates down the length of the channel The signal transduction concept by which the movement of the fluid is measured is discussed below.

In one embodiment, an RFID tag antenna design is integrated with the probe. The antenna's impedance is chosen such that as the fluid propagates down the probe, the flow manifests itself as a controlled drop in the backscatter signal strength of the tag. The probe-tag design and integration are discussed in more detail below. In one embodiment, both the probe and the tag were designed for use in the 902-928 MHz ISM band for UHF RFID operations as defined in North America. Other ISM bands may be used.

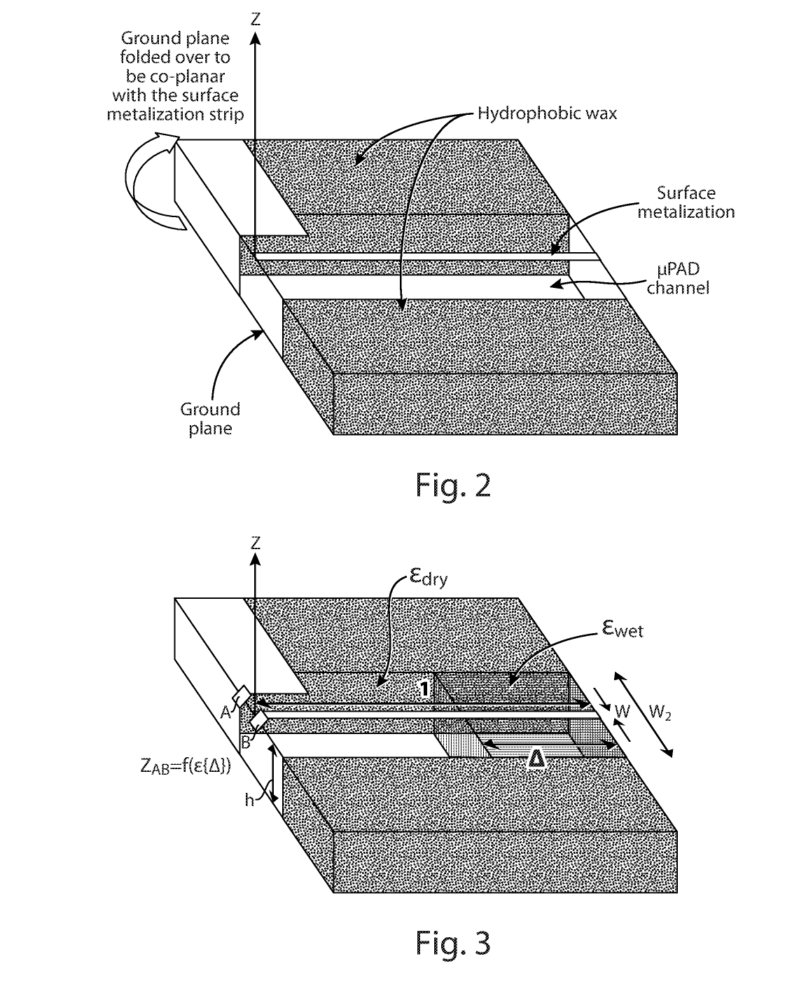

As seen in FIG. 2, in one embodiment, the probe includes a surface metallization strip and a ground plane, both made of copper, separated by a dielectric membrane. The surface metallization strip is in electrical communication with the RFID tag antenna and is connected across the points A and B in FIG. 3. In one embodiment, the dielectric is the μ-PAD paper. The hydrophobic wax permeates through the pores in the paper and contains the flow of fluid in the μ-PAD channel. Also, as illustrated in FIG. 2, the ground plane is folded so that the ground plane is co-planar with the surface metallization strip.

The impedance Z_{AB}, illustrated in FIG. 3, of the probe is dependent on several parameters such as the length of the metallization strip (1), the width of the strip (w), the width of the μ-PAD channel (w2), the wireless carrier frequency, and the height of the channel (h). Z_{AB} is also dependent on the relative dielectric permittivity (εᵣ) of the μ-PAD channel. For fixed geometrical dimensions, Z_{AB} is a function of the dielectric permittivity:

Z_{AB}=f(εᵣ) (1)

As fluid flows up the μ-PAD channel, illustrated by Δ in FIG. 3, it changes the value of εᵣ and therefore the Z_{AB}:

ΔZ_{AB}=f(Δεᵣ) (2)

The flow of fluid in the paper channel is therefore converted to a change in the electrical characteristics (the impedance) of the probe structure. This change in impedance is related to a change in backscatter signal strength of a passive UHF RFID tag as discussed below.

The passive UHF RFID tag generally includes two components: an antenna and an integrated circuit (IC). The antenna serves as a portion of a transceiver and serves the dual purpose of scavenging power from the RFID reader to power the IC and for sending RFID tag ID information back to the RFID reader via modulated backscatter. The magnitude of the backscatter power received by the reader, P_{BS|reader}, is given by:

P_{BS|reader}=Pₜ(G_{tag}Gᵣ)²(λ/4πd)⁴τ (3)

where Pₜ is the power transmitted by the reader antenna, Gᵣ and G_{tag} are the reader and tag antenna gains, d is the separation between the reader and tag antennas, λ is the wavelength of the carrier and τ is a measure of the impedance match between the antenna and the terminal load connected to the antenna. τ is given by:

τ=(4RₐRₗ)/(|Zₐ+Zₗ|)² (4)

where Rₐ and Rₗ are the effective resistance of the antenna and terminal load and Zₐ and Zₗ are the impedance of the antenna and terminal load, respectively. Typically, the terminal load is the tag IC and thus Zₗ=Z_{c} where Z_{c}=R_{c}+jX_{c} is the impedance of the tag IC.

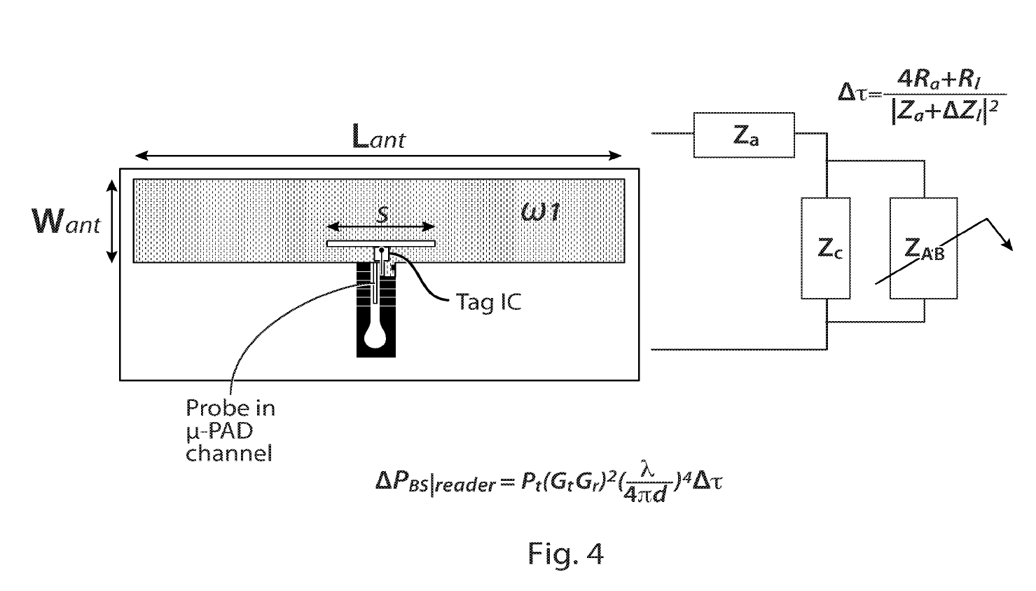

In one embodiment of the design shown in FIG. 4A, the probe is connected across the tag IC. As shown in the circuit model of the sensor FIG. 4B, the probe's impedance, Z_{AB} serves as a parasitic parallel impedance to Z_{c}. Therefore, from equation 4 above:

Zₗ=(Z_{c}Z_{AB})/(Z_{c}+Z_{AB}) (5)

Relating equation 2 with equations 4 and 5, τ is also a function of the flow of fluid in the paper channel:

Δτ=(4RₐRₗ)/(|Zₐ+ΔZₗ|)² (6)

From equation 6 and equation 3, the change in P_{BS reader} can be computed as:

ΔP_{BS|reader}=Pₜ(G_{tag}Gᵣ)²(λ/4πd)⁴Δτ (7)

In one embodiment, the RFID antenna as shown in FIG. 4 is an embedded T-match antenna. The antenna dimensions Lₐₙₜ, Wₐₙₜ, s and w1 are chosen so that τ is 1, and thus P_{BS|reader} is maximized, when there is no fluid in the channel As fluid propagates through the channel, τ is reduced per equation 6 and P_{BS|reader} is minimized when the channel is full of fluid.

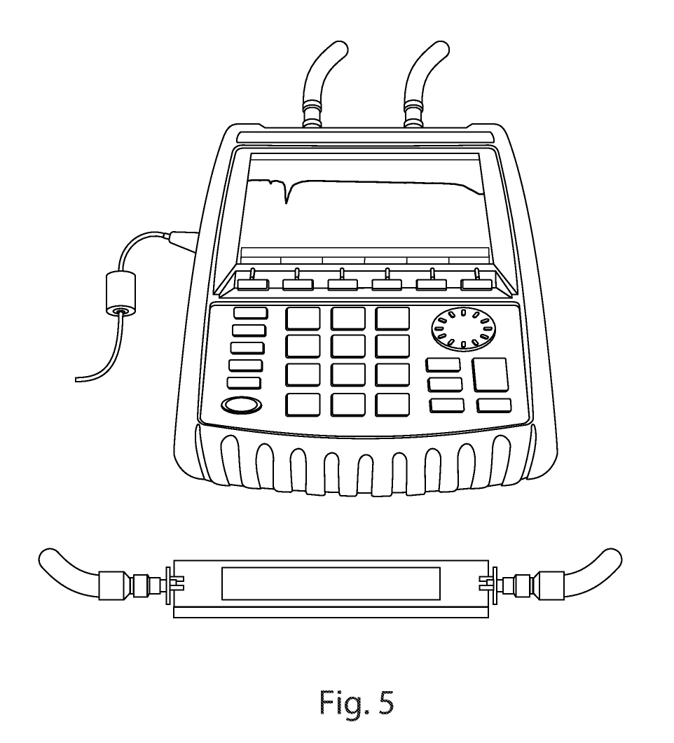

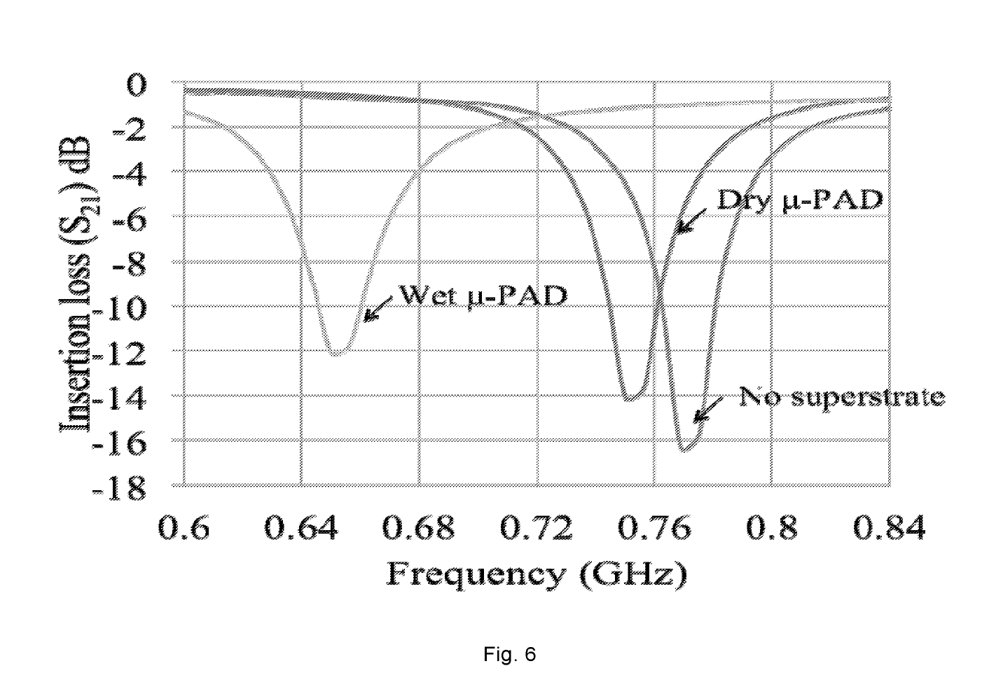

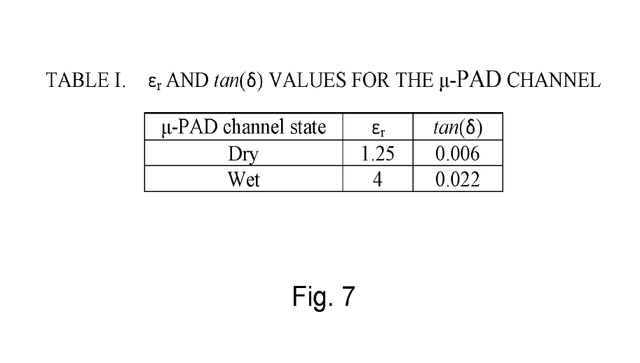

The first step in the design process is therefore to determine εᵣ loss tangent, tanδ, of the μ-PAD paper material in the dry and wet state. This is done by applying the μ-PAD paper material as a superstrate to a high-Q SIR (Stepped Impedance Resonator) filter (see FIG. 5). By observing the change in frequency dependence of the filter's insertion loss, S21, when dry and wet paper is applied as a superstrate (see FIG. 6), it is possible to compute εᵣ and tanδ of the wet and dry paper (see FIG. 7). A detailed theory of the dielectric characterization of the μ-PAD material based on this approach is described in the literature.

In the second step, the values of εᵣ are used to simulate the change in Z_{AB}. Referring to FIG. 3, in one embodiment, the following dimensions were used for fabrication simplicity and manageable probe size: w=0.5 mm, w2=4.5 mm. h=0.2 mm is the thickness of the μ-PAD paper. l was varied from 10-80 mm. Z_{AB} dry is then computed using ANSYS HFSS simulation software (Canonsburg, Pa., USA). The embedded T-match antenna dimensions were chosen so that Zₐ is impedance matched to Z_{l\dry} where:

Z_{l\dry}=(Z_{c}Z_{AB\dry})/(Z_{c}+Z_{AB\dry}) (8)

In one embodiment, an Alien Higgs 3 RFID IC (San José, Calif., USA) was used and has an experimentally determined value of Z_{c}=18−164i at 915 MHz.

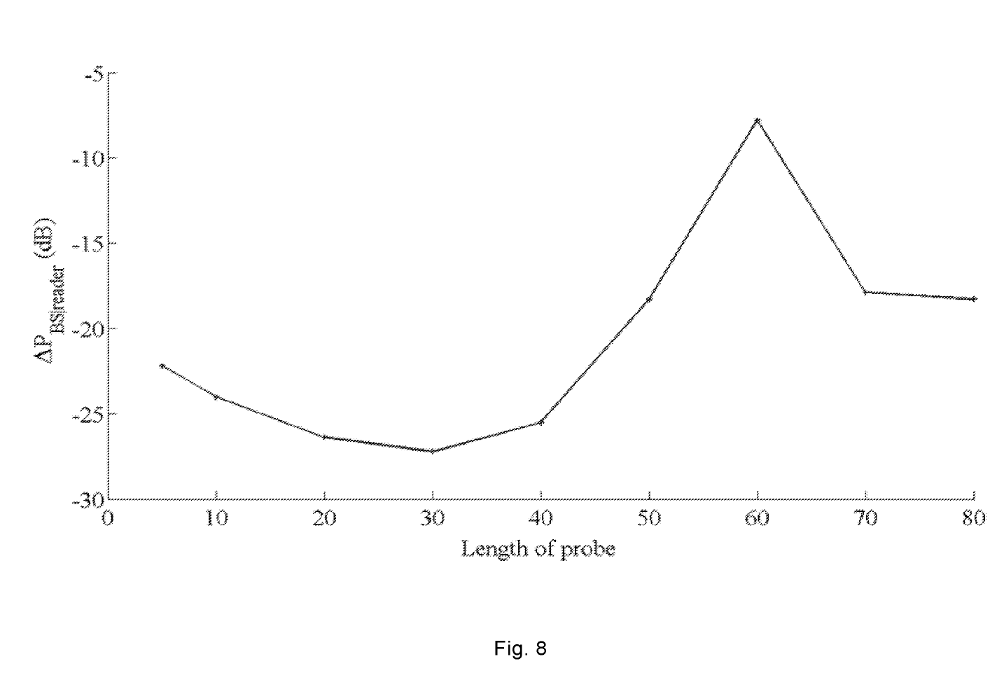

Using the table in FIG. 7 and the HFSS software, the probe impedance, Z_{AB|wet}, is computed when the μ-PAD is completely wet. Given the value of Zₐ, the expected change in τ and thus ΔP_{BS|reader} from Eq. 7 is computed. FIG. 8 illustrates the computed change ⁱ n ΔP_{BS|reader} as l is varied from 10 to 80 mm. As can be seen from the figure, a difference of at least 8 dB between the wet and dry states exists for all probe lengths. In one embodiment, l=10 mm was chosen since it offered a drop of 24 dB while keeping the net size of the probe small.

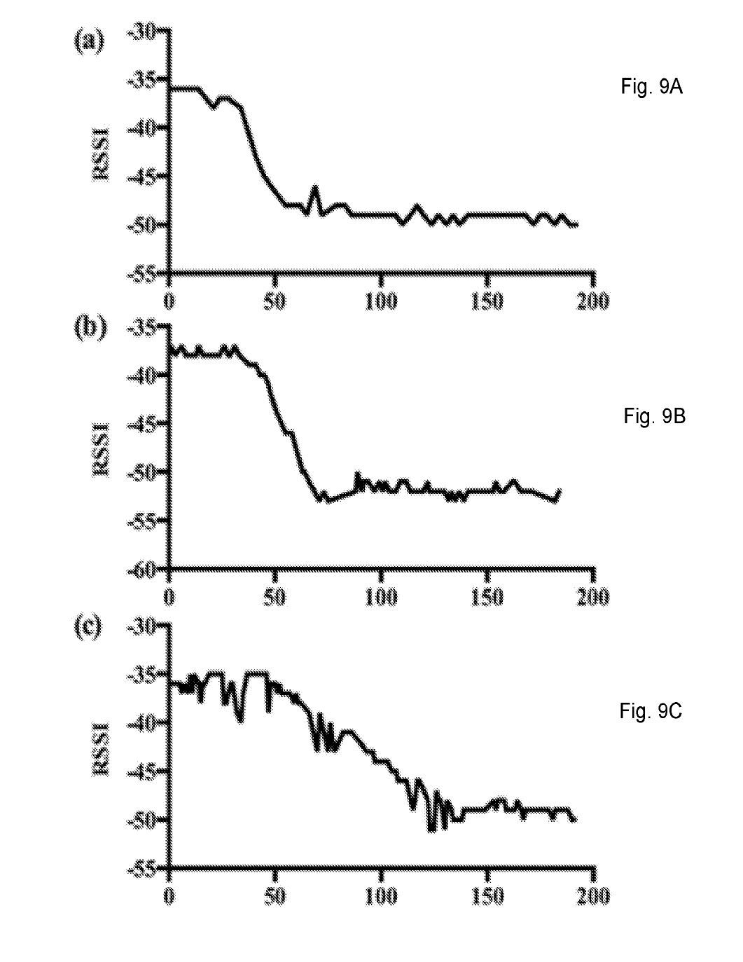

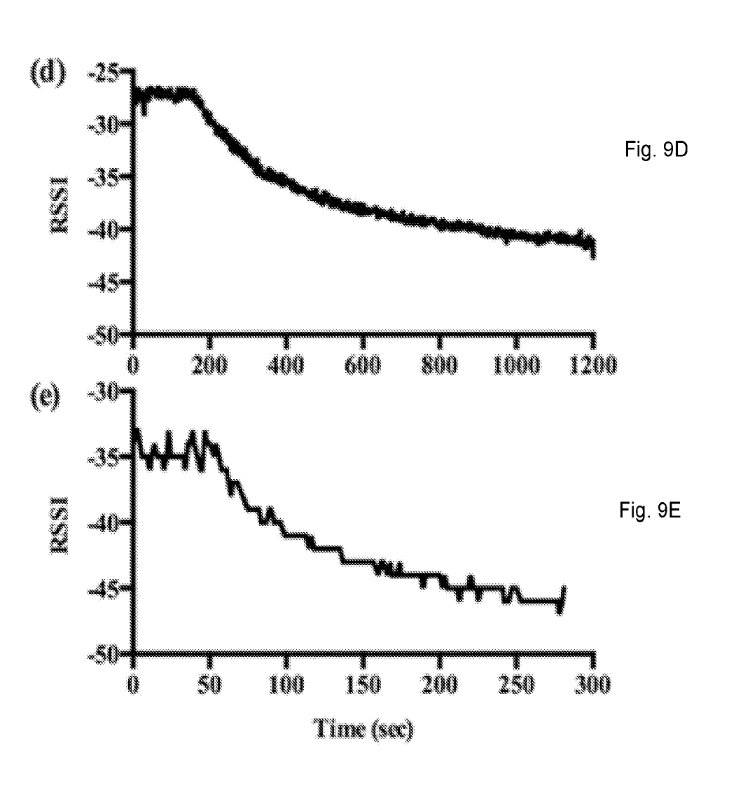

The backscatter signal change produced by wetting of the paper-based microfluidic device channel was evaluated for different fluids and different types of paper. To analyze the effect of different types of fluids in the device, 10 μL of distilled water (DIH₂O), 1X phosphate buffer saline (1X PBS) and human plasma were added to the sample port, S (see FIG. 1) and the change in the backscatter signal was monitored until the fluid had propagated all the way through the channel C (see FIGS. 9A-9C). FIG. 9 depicts the RFID tag backscatter signal drop with different fluids and paper materials. RFID tags were attached to paper-based assays constructed of nitrocellulose (9A-9C), Whatman 1 (9D) and Whatman 4 (9E) membranes. 10 μL of DIH₂O (9A), 10 μL of 1X PBS (9B), 10 μL of plasma (9C) were added to the nitrocellulose membranes. 20 μL of plasma was added to the Whatman membranes (9D and 9E). All fluids were able to create a significant drop in the backscatter signal as they flowed through the channel beneath the copper probe. However, because DIH₂O and 1X PBS (phosphate-buffered saline) are much less viscous than plasma, they flowed through the channel faster than plasma which is evidenced by the faster drop in the backscatter signal on the plots.

The ability of the RFID tag to track fluid flow in different types of materials was next measured. Commonly, lateral flow assays utilize nitrocellulose membrane, while paper-based flow-through assays, which are becoming more popular due to a lower cost of materials, employ Whatman 1 and Whatman 4 filtration papers. To select what materials might be used, the change in backscatter signal as plasma flowed up the hydrophilic channels created in these different materials was characterized. Each type of material demonstrated significant signal drop following wetting of the channel, indicating that each material is conducive to being used in devices for RFID tags (see FIGS. 9C-9E). As nitrocellulose membrane allowed for faster fluid flow and has a smaller void volume, one embodiment used this material.

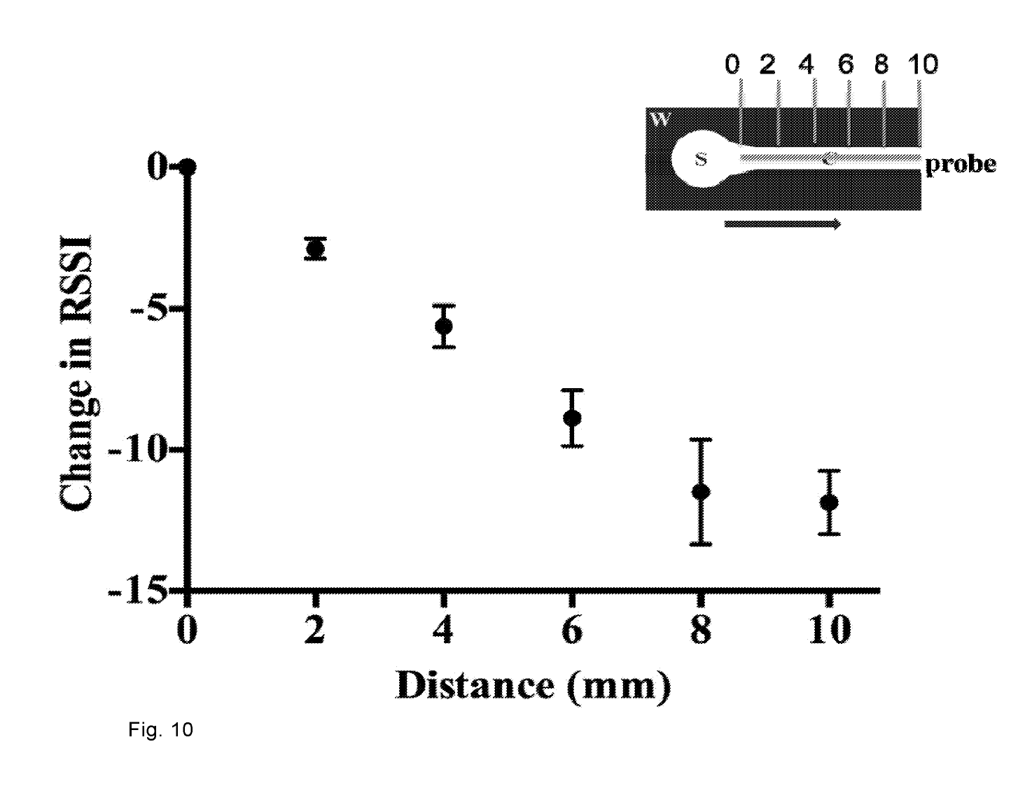

In order to use a timing assay using the RFID tag, it is important to characterize the change in the backscatter drop as fluid flowed up the probe. For this characterization, human plasma was added to the sample port of eight devices and the drop in signal as the fluid flowed in the channel under each 2 mm segment of the copper probe was measured. It was found that most of the backscatter signal drop occurred when fluid flowed underneath the first 8 mm of the copper probe, while only a small drop occurred over the last 2 mm of the probe (see FIG. 10). Moreover, the drop in signal over the first 8 mm of the probe followed a linear pattern (y=−1.45x+0.025, R²=0.9490) allowing the prediction of how much signal drop can be achieved when the channel under each section of the probe is wetted.

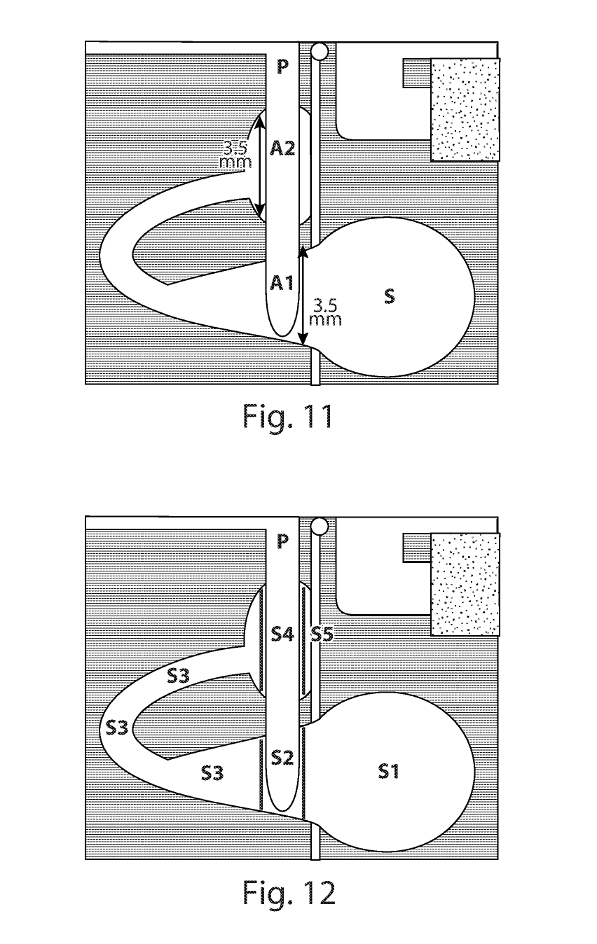

In order to measure the speed of flow of the plasma in the paper-based microfluidic assay, two separate areas of measurement on the assay were required. Utilizing the start and end points of the channel for measurements permitted the longest flow path and greatest differentiation of hematocrit values. In one embodiment, the paper-based device for hematocrit measurement was designed as an arch to permit two distinct areas of measurement along the linear 10 mm RFID tag probe (labeled as A1 and A2 in FIG. 11). Because testing had demonstrated that the most sensitive area of the probe was the first 8 mm (see FIG. 10), measurement focused the start and end points of the channel in this region.

The paper portion of this arched hematocrit assay device included a sample port, where a specified volume of blood is added, and a contiguous hydrophilic channel of varying thickness along its length (see FIG. 11). Similar to the straight channel design, hydrophobic barriers surrounding the hydrophilic channels were created by printing a layer of wax on top of the nitrocellulose and melting it evenly. Careful selection of the membrane porosity, assay geometry and plasma separation buffer components produced robust separation of the plasma into the adjacent hydrophilic channel To optimize the differentiation of flow times for samples with lower hematocrit, a range of sample port diameters and channel lengths were used. For the final design of one embodiment of the hematocrit sensor, a sample port of diameter of 5.5 mm and a channel length of 16 mm was selected.

Following wax printing, 1.70 μL of plasma separation buffer was added to the center of the sample port of each assay and allowed to spread out radially to fill the sample port area. Assays were dried at 65° C. for 2 minutes and stored at room temperature until integrated with the RFID tag. The 10 mm probe of each antenna was aligned across the paper-based hematocrit assay as specified above. The first 3.5 mm of the tip of the probe overlapped with the channel as it exited the sample port area. This allowed plasma to be detected immediately after it is separated from blood and entered the curved channel The next 4.5 to 8 mm of the probe was placed over the end of the curved channel (see FIG. 10). This allowed plasma to be detected when it reached the end of the channel. Similar to the linear paper-assay design, the ground plane of the microstrip probe was folded around the edge of the paper assay and attached to the back of the paper assay. The paper assay and the microstrip probe, excluding the sample port area, were then sealed with laminate (Fellowes, Itasca, Ill., USA). Finished devices were stored at room temperature in a variable humidity environment until used.

Devices consisting of a paper-based assay and attached RFID tag were manufactured and tested using human blood (Innovative Research, Inc., Novi, Mich., USA). The initial measurement of hematocrit was performed by filling a capillary tube with blood and sealing one end with clay. The capillary tube was centrifuged at 10,500 RPM for 5 minutes using a ZIPocrit centrifuge (LW Scientific, Lawrenceville, Ga., USA). The percent hematocrit was estimated using a microhematocrit centrifuge. To create hematocrit samples from 20% to 50%, plasma was then either added to the blood (for lower hematocrit values) or removed from the blood (for higher hematocrit values).

The hematocrit sensors were placed at a distance of 35 cm from the RFID reader and the reader was programed to make four readings per second with a maximum transmit power of 36 dBm. Thirty seconds after RFID readings were initiated, 15 μL of whole blood were added to each device. The movement of plasma from the sample port down the channel was evaluated both visually and electronically. Visual analysis of the plasma flow time utilized the point at which the plasma passed completely under the first probe area (A1 in FIG. 11) and the point at which the plasma first touched the metallization corresponding to the second probe area (A2 in FIG. 11). The hematocrit sensors were monitored for up to 15 minutes, until the plasma had completely filled the end of the channel.

To fine-tune the data and reduce the noise in the backscatter signal due to multipath and frequency hopping effects, the average backscatter signal value over every 2 seconds was determined. FIG. 12 depicts the start points of zones S1-S5 on the assay device. For each device, graphs relating the backscatter signal to the read time were created. Each graph has five specific stages: (S1) an initial signal produced by the completely dry membrane, (S2) a sharp decrease in the backscatter signal as plasma passes the first area, A1, under the probe, (S3) a signal plateau as plasma flows around the curved channel, (S4) a second sharp drop in the signal as plasma first touches and flows under the metallization of the second area, A2, under the probe, and (S5) a final plateau after the channel has been completely filled and the maximum signal drop achieved.

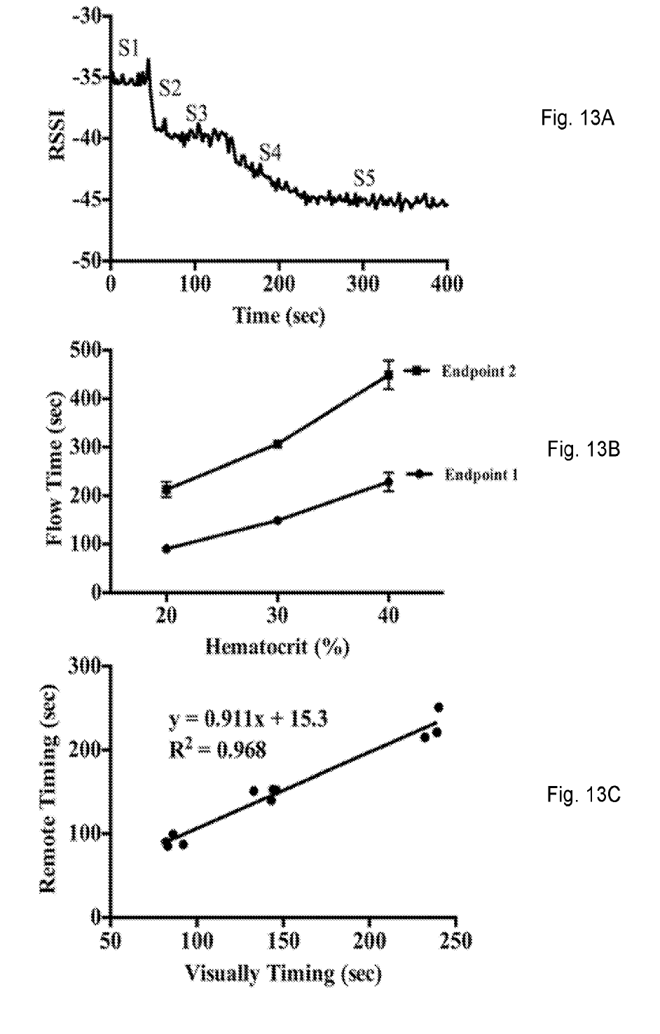

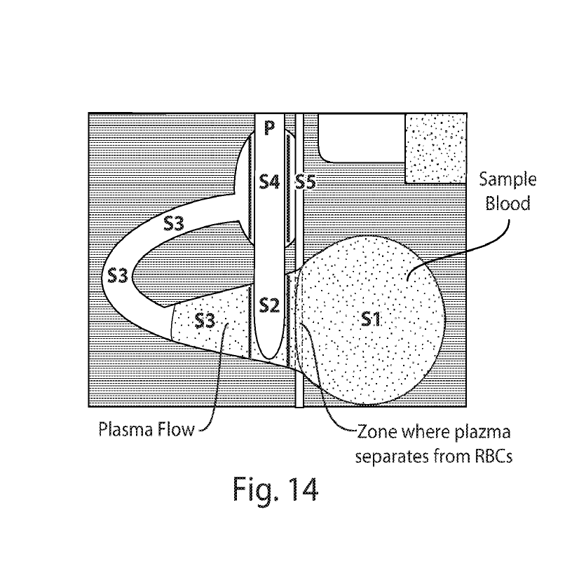

FIG. 13 shows an assay device seeded with a blood sample. FIG. 13A illustrates the corresponding change in tag RSSI (Received Signal Strength Indicator) power measurements versus time for blood with 30% hematocrit. The graph may be divided into five stages labeled S1-S5. FIG. 13B depicts the average flow times of blood with different hematocrit percentages as determined utilizing endpoints 1 and 2 with remote RFID sensing. Tests were repeated four times for each hematocrit percentage level. FIG. 13C depicts the comparison of flow times as determined visually and utilizing remote RFID sensing with endpoint 1 of FIG. 13B.

The start time of plasma flow was determined to be the point at which stage 2 began. End times for plasma flow were calculated in two manners: (1) the beginning of S4 and (2) the beginning of S5. To initially test the efficacy of the device, blood samples with hematocrit levels from 20% to 50% were tested in quadruplicate and the average plasma flow times calculated. Using RFID enabled remote sensing, blood samples with hematocrit levels of 20, 30, 40 and 50 percent had flow times of 90, 149, 229 and >900 seconds (endpoint 1 measurements), respectively (see FIG. 13B). Coefficients-of-variation were below 9% and analysis of data utilizing the Students T-test demonstrated significant differences between the hematocrit levels (p-value<0.001 for 20% vs. 30% hematocrit, p-value=0.014 for 30% vs. 40% hematocrit). Notably, data for blood with 50% hematocrit was not included in these calculations, as the assay took greater than 15 minutes to run. Similar results were found when flow times were measured with endpoint 2. In one embodiment, the data was recorded and an estimate of time was made manually. However, it is not necessary that this process be done manually. In another embodiment, the RFID reader is programmed to record start and stop time based on the knowledge of the shape of the curve of FIG. 13 (A).

In order for remote sensing utilizing RFID tags to be useful for time-based point-of-care assays, it is essential that the results from the RFID tag closely match the results achieved visually. Comparing the flow times achieved by these two different data acquisition methods for the hematocrit measurements, a linear relationship was determined (y=0.911x+15.3, R²=0.968) between visual measurements and RFID measurements made at endpoint 1 (see FIG. 12C). Minor variations in manufacturing tolerances within a class do not have a significant effect on being able to separate the classes. Here, the 15.3 second offset may be attributed to the different start points for the visual and RFID methods, beginning at S2 and S3, respectively. Measurements made at endpoint 2, however, were not similar to visual time measurements as they were offset by the amount of time that it took for the plasma to fill the channel area underneath the probe. Together, these indicate that careful attention must be paid to the design of the timing assay to ensure a through understanding of the time values achieved.

Although the use of this device in measuring blood anomalies such as anemia has been discussed, it may be used to measure components of many fluids that can be separated by fluid flow.

Unless otherwise indicated, all numbers expressing lengths, widths, depths, or other dimensions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Any specific value may vary by 20%.

The terms “a,” “an,” “the,” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventor for carrying out the spirit of the present disclosure. Of course, variations on these described embodiments will become apparent to those of ordinary skill in the art upon reading the foregoing description. The inventor expects skilled artisans to employ such variations as appropriate, and the inventor intends for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of measuring hematocrit comprising the steps of:
providing an assay device comprising:
an RFID tag;
a micro-PAD defining a sample port and a channel, the micro-PAD in electrical communication with the RFID tag;
measuring, using an RFID reader the change of impedance of the RFID tag over time; and
determining the hematocrit of the sample in response to the time change of impedance.

2. A system for measuring the hematocrit of a sample, the system comprising:
an assay device comprising:
an RFID tag;
a micro-PAD defining a sample port and a channel, the micro-PAD in electrical communication with the RFID tag; and
an RFID reader,
wherein the RFID reader measures the change of impedance of the RFID tag over time; and determines the hematocrit of the sample in response to the time change of impedance.

3. A wireless blood anomaly detection system comprising:
an RFID tag comprising an RFID chip and an RFID antenna; and
a blood probe comprising:
a substrate defining a port in fluid communication with a channel, the channel including a metallic strip and a ground plane,
wherein the metallic strip is in electrical communication with the RFID antenna,
wherein the port is configured to accept a blood sample, and
wherein the channel has an impedance which changes as the blood sample moves from the port and through the channel, and
an RFID reader generating a carrier signal and configured to monitor the change in back scatter signal of the carrier signal, as reflected back by the RFID tag, as blood moves down the channel.

4. The wireless blood anomaly detection system of claim 3 wherein the RFID reader measures the time it takes for blood to move down the channel in response to a change in back scatter frequency and generates an hematocrit value in response to the time of blood movement.

5. The wireless blood anomaly detection system of claim 3 wherein the RFID reader measures the time it takes for blood to move down the channel in response to a change in signal strength and generates an hematocrit value in response to the time of blood movement.

6. A wireless blood anomaly detection sensor comprising:
an RFID tag comprising an RFID chip and an RFID antenna; and
a blood probe comprising:
a substrate defining a port in fluid communication with a channel, the channel including a metallic strip and a ground plane,
wherein the metallic strip is in electrical communication with the RFID antenna,
wherein the port is configured to accept a blood sample, and
wherein the channel has an impedance which changes as the blood moves from the port and through the channel.

7. A method for blood anomaly detection comprising:
providing an RFID tag comprising an RFID chip and an RFID antenna; and a blood probe, the blood probe comprising:
a substrate defining a port in fluid communication with a channel, the channel including a metallic strip and a ground plane,
wherein the metallic strip is in electrical communication with the RFID antenna,
wherein the port is configured to accept a blood sample, and
wherein the channel has an impedance which changes as the blood sample moves from the port and through the channel;
generating a carrier signal with an RFID reader; and
monitoring with the RFID reader, the change in back scatter of the carrier signal as blood moves down the channel.

8. The method for blood anomaly detection of claim 7 wherein the backscatter measurement comprises measuring a change in signal strength.

9. The method for blood anomaly detection of claim 7 wherein the backscatter measurement comprises measuring a change in the frequency response of the RFID tag received by the RFID reader.

10. A wireless fluid component detection system comprising:
an RFID tag comprising an RFID chip and an RFID antenna; and
a fluid probe comprising:
a substrate defining a port in fluid communication with a channel, the channel including a metallic strip and a ground plane,
wherein the metallic strip is in electrical communication with the RFID antenna,
wherein the port is configured to accept a fluid sample, and
wherein the channel has an impedance which changes as the fluid sample moves from the port and through the channel, and
an RFID reader generating a carrier signal and configured to monitor the change in back scatter signal of the carrier signal, as reflected back by the RFID tag, as fluid moves down the channel.

11. The wireless fluid component detection system of claim 10 wherein the RFID reader measures the time it takes for a fluid sample to move down the channel in response to a change in RFID tag back scatter frequency and generates a component concentration value in response to the time of fluid movement.

12. The wireless fluid component detection system of claim 10 wherein the RFID reader measures the time it takes for a fluid sample to move down the channel in response to a change in signal strength and generates a component concentration value in response to the time of fluid movement.

13. A method for fluid component detection comprising:
providing an RFID tag comprising an RFID chip and an RFID antenna; and a fluid probe, the fluid probe comprising:
a substrate defining a port in fluid communication with a channel, the channel including a metallic strip and a ground plane,
wherein the metallic strip is in electrical communication with the RFID antenna,
wherein the port is configured to accept a fluid sample, and
wherein the channel has an impedance which changes as the fluid sample moves from the port and through the channel;
generating a carrier signal with an RFID reader; and
monitoring with the RFID reader, the change in back scatter of the carrier signal as fluid moves down the channel

14. The method for fluid component detection of claim 13 wherein the backscatter measurement comprises measuring a change in signal strength.

15. The method for fluid component detection of claim 13 wherein the backscatter measurement comprises measuring a shift or change in the frequency response of the RFID tag received by the RFID reader.

16. A wireless fluid component detection sensor comprising:
an RFID tag comprising an RFID chip and an RFID antenna; and
a fluid probe comprising:
a substrate defining a port in fluid communication with a channel, the channel including a metallic strip and a ground plane,
wherein the metallic strip is in electrical communication with the RFID antenna,
wherein the port is configured to accept a fluid sample, and
wherein the channel has an impedance which changes as the fluid moves from the port and through the channel.
